SEQUENCE LISTING (20 sequences)

SEQ ID NO: 1 - DNA, 1146 bp, Moorena producens
  source 1..1146: mol_type = genomic DNA; strain = Moorena producens PAL-8-15-08-1
atgtcggaaaaaattgttaattcctggaatgaatgggatgaattggaagaaatggtggtaggaattgcagactatgctagctttgaaccaaaagaaccagggaatcatccgaaattaagaaatcaaaatttagcggaaatcattcctttccccagtggacctaaagaccctaaagtccttgaaaaagctaatgaagaattaaatggactggcttatttattaaaagaccacgatgtgatagtaagaagacccgaaaaaattgattttactaaatctctaaaaacaccttactttgaagttgcaaatcaatactgtggagtctgtcctcgggatgtcatgattacctttgggaatgaaatcatggaagcgactatgtcgaagagagctagattttttgaatacttaccttaccggaaattggtctatgaatattggaataaagacgagcatatgatttggaatgctgcgcctaaaccgactatgcaggatagtatgtatctagagaatttctgggagctgtctttagaagaacgatttaagcgtatgcatgattttgaattttgtattacacaagatgaagtaatttttgatgcggctgactgtagcagattaggaaaggatatattagttcaggaatcgatgacaacaaatagaacaggaattcggtggttaaaaaagcacctagaaccaagaggatttcgggttcaccctgttcattttccccttgattttttcccctcacacattgactgtacgtttgttcctttgcgaccaggtcttattttgacaaaccctgaaagacctatacgggaagaggaggagaagatttttaaagagaatggctgggagttgatcacagttcctcaaccgacttgctcgaatgatgaaatgccaatgttttgccagtccagtaagtggttgtcaatgaatgttctgagtatatcaccgacaaaggttatctgtgaggaaagagaaaaacctctccaagaattgttggataagcatggatttgaggtttttcctttaccctttagacatgtctttgaatttggggggtcttttcattgtgcaacttgggatattcgccgaaaaggtgagtgtgaagattatttaccaaatttaaactatcaaccgatttgtggttaa

SEQ ID NO: 2 - protein, 381 aa, Moorena producens
  source 1..381: note = strain: Moorena producens PAL-8-15-08-1
MSEKIVNSWNEWDELEEMVVGIADYASFEPKEPGNHPKLRNQNLAEIIPFPSGPKDPKVLEKANEELNGLAYLLKDHDVIVRRPEKIDFTKSLKTPYFEVANQYCGVCPRDVMITFGNEIMEATMSKRARFFEYLPYRKLVYEYWNKDEHMIWNAAPKPTMQDSMYLENFWELSLEERFKRMHDFEFCITQDEVIFDAADCSRLGKDILVQESMTTNRTGIRWLKKHLEPRGFRVHPVHFPLDFFPSHIDCTFVPLRPGLILTNPERPIREEEEKIFKENGWELITVPQPTCSNDEMPMFCQSSKWLSMNVLSISPTKVICEEREKPLQELLDKHGFEVFPLPFRHVFEFGGSFHCATWDIRRKGECEDYLPNLNYQPICG

SEQ ID NO: 3 - DNA, 1362 bp, synthetic construct
  source 1..1362: mol_type = other DNA
ggtctccatccgcgtcggagacgaagtaattgacggcagcacctcgggcaaactcgagcgtctgcgggcaagcttcgcataaagacacgacgaattagacaacattagtaatgctggaagaaacaaccgagagcaggaagaacatgtcagaaaagattgttaactcctggaacgaatgggacgaacttgaagaaatggtggttggcatcgcggactacgcttccttcgagccaaaagaaccaggtaaccaccctaagctacgcaaccagaacctggccgagatcatcccattcccatccggcccaaaggacccaaaggttctcgaaaaagcaaacgaagagctgaacggcctcgcatacctcctaaaggaccacgatgttatcgtccgccgcccagaaaaaattgatttcactaagtcccttaaaaccccatacttcgaggtcgctaaccagtactgcggcgtttgcccacgcgacgttatgattaccttcggtaatgagattatggaggctaccatgagcaagcgtgcacgcttcttcgagtacttgccataccgtaagctcgtctacgagtactggaacaaggatgagcatatgatctggaacgctgctccaaagcctacgatgcaggactccatgtacttggagaacttctgggagctgtccctcgaggagcgcttcaagcgcatgcacgacttcgaattttgcattacccaggacgaggttatcttcgacgcagctgactgctcccgcctgggcaaggacatcctggtccaggagtccatgaccaccaaccgcaccggcattcgttggttgaagaagcacctcgaacctcgcggtttccgcgttcacccagtccacttccctctcgacttcttcccttctcacattgactgcactttcgtccctctccgtcccggcctcatccttaccaacccagagcgcccaatccgcgaagaagaagaaaagatcttcaaggagaacggctgggagcttatcaccgtcccacaacctacctgctccaacgacgaaatgcctatgttctgccagtcctcgaagtggctcagcatgaacgtcctttctatctccccaacaaaggttatttgcgaagagcgtgaaaagccactacaggaactcctggacaagcacggcttcgaagtcttcccactcccattccgccacgttttcgaattcggcggctcctttcactgcgccacttgggacatccgccgcaagggcgaatgcgaagactacctcccaaacctgaactaccagcctatctgcggctaataagctcggtacccggggatccgacgtcgcgttaacttaaggtaccgaattctaagcttcacaaaaagagacc

SEQ ID NO: 4 - DNA, 3716 bp, synthetic construct
  source 1..3716: mol_type = other DNA
agcaacaccttcttcacgaggcagatctcagcgcccccccccccctagcttgtctacgtctgatgctttgaatcggacggacttgccgatcttgtatgcggtgatttttccctcgtttgcccactttttaatggtggccggggtgagagctacgcgggcggcgacctgctgcgctgtgatccaatattcggggtcgttcactggttcccctttctgatttctggcatagaagaacccccgtgaactgtgtggttccgggggttgctgatttttgcgagacttctcgcgcaattccctagcttaggtgaaaacaccatgaaacactagggaaacacccatgaaacacccattagggcagtagggcggcttcttcgtctagggcttgcatttgggcggtgatctggtctttagcgtgtgaaagtgtgtcgtaggtggcgtgctcaatgcactcgaacgtcacgtcatttaccgggtcacggtgggcaaagagaactagtgggttagacattgttttcctcgttgtcggtggtggtgagcttttctagccgctcggtaaacgcggcgatcatgaactcttggaggttttcaccgttctgcatgcctgcgcgcttcatgtcctcacgtagtgccaaaggaacgcgtgcggtgaccacgacgggcttagcctttgcctgcgcttctagtgcttcgatggtggcttgtgcctgcgcttgctgcgcctgtagtgcctgttgagcttcttgtagttgctgttctagctgtgccttggttgccatgctttaagactctagtagctttcctgcgatatgtcatgcgcatgcgtagcaaacattgtcctgcaactcattcattatgtgcagtgctcctgttactagtcgtacatactcatatttacctagtctgcatgcagtgcatgcacatgcagtcatgtcgtgctaatgtgtaaaacatgtacatgcagattgctgggggtgcagggggcggagccaccctgtccatgcggggtgtggggcttgccccgccggtacagacagtgagcaccggggcacctagtcgcggataccccccctaggtatcggacacgtaaccctcccatgtcgatgcaaatctttaacattgagtacgggtaagctggcacgcatagccaagctaggcggccaccaaacaccactaaaaattaatagttcctagacaagacaaacccccgtgcgagctaccaactcatatgcacgggggccacataacccgaaggggtttcaattgacaaccatagcactagctaagacaacgggcacaacacccgcacaaactcgcactgcgcaaccccgcacaacatcgggtctaggtaacactgaaatagaagtgaacacctctaaggaaccgcaggtcaatgagggttctaaggtcactcgcgctagggcgtggcgtaggcaaaacgtcatgtacaagatcaccaatagtaaggctctggcggggtgccataggtggcgcagggacgaagctgttgcggtgtcctggtcgtctaacggtgcttcgcagtttgagggtctgcaaaactctcactctcgctgggggtcacctctggctgaattggaagtcatgggcgaacgccgcattgagctggctattgctactaagaatcacttggcggcgggtggcgcgctcatgatgtttgtgggcactgttcgacacaaccgctcacagtcatttgcgcaggttgaagcgggtattaagactgcgtactcttcgatggtgaaaacatctcagtggaagaaagaacgtgcacggtacggggtggagcacacctatagtgactatgaggtcacagactcttgggcgaacggttggcacttgcaccgcaacatgctgttgttcttggatcgtccactgtctgacgatgaactcaaggcgtttgaggattccatgttttcccgctggtctgctggtgtggttaaggccggtatggacgcgccactgcgtgagcacggggtcaaacttgatcaggtgtctacctggggtggagacgctgcgaaaatggcaacctacctcgctaagggcatgtctcaggaactgactggctccgctactaaaaccgcgtctaaggggtcgtacacgccgtttcagatgttggatatgttggccgatcaaagcgacgccggcgaggatatggacgctgttttggtggctcggtggcgtgagtatgaggttggttctaaaaacctgcgttcgtcctggtcacgtggggctaagcgtgctttgggcattgattacatagacgctgatgtacgtcgtgaaatggaagaagaactgtacaagctcgccggtctggaagcaccggaacgggtcgaatcaacccgcgttgctgttgctttggtgaagcccgatgattggaaactgattcagtctgatttcgcggttaggcagtacgttctagattgcgtggataaggctaaggacgtggccgctgcgcaacgtgtcgctaatgaggtgctggcaagtctgggtgtggattccaccccgtgcatgatcgttatggatgatgtggacttggacgcggttctgcctactcatggggacgctactaagcgtgatctgaatgcggcggtgttcgcgggtaatgagcagactattcttcgcacccactaaaagcggcataaaccccgttcgatattttgtgcgatgaatttatggtcaatgtcgcgggggcaaactatgatgggtcttgttgttgacaatggctgatttcatcaggaatggaactgtcatgctgttatgtgcctggctcctaatcaaagctggggacaatgggttgccccgttgatctgatctagttcggattggcggggcttcactgtatctgggggtggcatcgtgaatagattgcacaccgtagtgggcagtgtgcacaccatagtggccatgagcaccaccacccccagggacgccgacggcgcgaagctctgcgcctggtgcggctcggagatcaagcaatccggcgtcggccggagccgggactactgccgccgctcctgccgccagcgggcgtacgaggcccggcgccagcgcgaggcgatcgtgtccgccgtggcgtcggcagtcgctcgccgagatacgtcacgtgacgaaatgcagcagccttccattccgtcacgtgacgaaactcgggccgcaggtcagagcacggttccgcccgctccggccctgccggacccccggcatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatccgagtccaatgtgtccgtgttcacctcagcggccgcaccggtcggggtaccttgacaaatactttccaatggggtacaatggcgcagtgccatccactccgaggttgataccagcctcctcggtggaatgatcatccgcgtcggagacgaagtaattgacggcagcacctcggggagacctcaaatggtctcgcgttaacttaaggtaccgaattctaagcttcacaaaaaaaaaccccgcccctgacagggcggggttttttttactcagcatgtggatggccagtggtcggaccgagcgctttaattaattagcatggtgacacaagcacagtat

SEQ ID NO: 5 - DNA, 906 bp, Corynebacterium glutamicum
  source 1..906: mol_type = genomic DNA
atgaataaacagtccgctgcagtgttgatggtgatgggttccgccctatccctgcaatttggtgctgccattggaacgcagcttttccccctcaacggcccctgggctgtcacctctttaaggctgttcatcgcaggcttgatcatgtgcctggtgatccgcccgcgacttcgttcctggactaaaaaacaatggatcgccgtgctgctgttgggattatctcttggcggaatgaacagcctgttttacgcatccatcgaactcatcccgctgggtaccgccgtgaccattgagttcctcggccccctgattttctccgcggtgttagcccgcacgctgaaaaacggattgtgcgtggctttagcgtttctcggcatggcactactgggtatcgattccctcagcggcgaaacccttgacccactcggcgtcattttcgcagccgtcgcaggaatcttctgggtgtgctacatcctggcatcaaagaaaatcggccaactcatccccggaacaagcggcctggccgtcgcactgattatcggcgcagtggcagtatttccactgggtgctacacacatgggcccgattttccagaccccaaccctactcatcctggcgcttggcacagcacttctcgggtcgcttatcccctattcgctggaattatcggcactgcgccgactccccgcccccattttcagtattctgctcagcctcgaaccggcattcgccgccgccgtcggctggatcctgcttgatcaaacccccaccgcgctcaagtgggccgcgatcatccttgtcatcgcggccagcatcggcgtcacgtgggagcctaaaaagatgcttgtcgacgcgcccctccactcaaaatgcaacgcgaagaggcgagtacacacacctagttaa

SEQ ID NO: 6 - protein, 301 aa, Corynebacterium glutamicum
MNKQSAAVLMVMGSALSLQFGAAIGTQLFPLNGPWAVTSLRLFIAGLIMCLVIRPRLRSWTKKQWIAVLLLGLSLGGMNSLFYASIELIPLGTAVTIEFLGPLIFSAVLARTLKNGLCVALAFLGMALLGIDSLSGETLDPLGVIFAAVAGIFWVCYILASKKIGQLIPGTSGLAVALIIGAVAVFPLGATHMGPIFQTPTLLILALGTALLGSLIPYSLELSALRRLPAPIFSILLSLEPAFAAAVGWILLDQTPTALKWAAIILVIAASIGVTWEPKKMLVDAPLHSKCNAKRRVHTPS

SEQ ID NO: 7 - DNA, 42 bp, synthetic construct
  source 1..42: mol_type = other DNA
gtgtccgtgttcacctcagccgatctagtccttaaaggtttc

SEQ ID NO: 8 - DNA, 40 bp, synthetic construct
  source 1..40: mol_type = other DNA
caaggtaccccgaccggtgcgaagagcttccaaaaggtaa

SEQ ID NO: 9 - DNA, 1470 bp, Corynebacterium glutamicum
  source 1..1470: mol_type = genomic DNA
atgttgagttttgcgacccttcgtggccgcatttcaacagttgacgctgcaaaagccgcacctccgccatcgccactagccccgattgatctcactgaccatagtcaagtggccggtgtgatgaatttggctgcgagaattggcgatattttgctttcttcaggtacgtcaaatagtgacaccaaggtacaagttcgagcagtgacctctgcgtacggtttgtactacacgcacgtggatatcacgttgaatacgatcaccatcttcaccaacatcggtgtggagaggaagatgccggtcaacgtgtttcatgttgtaggcaagttggacaccaacttctccaaactgtctgaggttgaccgtttgatccgttccattcaggctggtgcgaccccgcctgaggttgccgagaaaatcctggacgagttggagcaatcccctgcgtcttatggtttccctgttgcgttgcttggctgggcaatgatgggtggtgctgttgctgtgctgttgggtggtggatggcaggtttccctaattgcttttattaccgcgttcacgatcattgccacgacgtcatttttgggaaagaagggtttgcctactttcttccaaaatgttgttggtggttttattgccacgctgcctgcatcgattgcttattctttggcgttgcaatttggtcttgagatcaaaccgagccagatcatcgcatctggaattgttgtgctgttggcaggtttgacactcgtgcaatctctgcaggacggcatcacgggcgctccggtgacagcaagtgcacgatttttcgaaacactcctgtttaccggcggcattgttgctggcgtgggtttgggcattcagctttctgaaatcttgcatgtcatgttgcctgccatggagtccgctgcagcacctaattattcgtctacattcgcccgcattatcgctggtggcgtcaccgcagcggccttcgcagtgggttgttacgcggagtggtcctcggtgattattgcggggcttactgcgctgatgggttctgcgttttattacctcttcgttgtttatttaggccccgtctctgccgctgcgattgctgcaacagcagttggtttcactggtggtttgcttgcccgtcgattcttgattccaccgttgattgtggcgattgccggcatcacaccaatgcttccaggtctagcaatttaccgcggaatgtacgccaccctgaatgatcaaacactcatgggtttcaccaacattgcggttgctttagccactgcttcatcacttgccgctggcgtggttttgggtgagtggattgcccgcaggctacgtcgtccaccacgcttcaacccataccgtgcatttaccaaggcgaatgagttctccttccaggaggaagctgagcagaatcagcgccggcagagaaaacgtccaaagactaatcagagattcggtaataaaaggtaa

SEQ ID NO: 10 - protein, 489 aa, Corynebacterium glutamicum
MLSFATLRGRISTVDAAKAAPPPSPLAPIDLTDHSQVAGVMNLAARIGDILLSSGTSNSDTKVQVRAVTSAYGLYYTHVDITLNTITIFTNIGVERKMPVNVFHVVGKLDTNFSKLSEVDRLIRSIQAGATPPEVAEKILDELEQSPASYGFPVALLGWAMMGGAVAVLLGGGWQVSLIAFITAFTIIATTSFLGKKGLPTFFQNVVGGFIATLPASIAYSLALQFGLEIKPSQIIASGIVVLLAGLTLVQSLQDGITGAPVTASARFFETLLFTGGIVAGVGLGIQLSEILHVMLPAMESAAAPNYSSTFARIIAGGVTAAAFAVGCYAEWSSVIIAGLTALMGSAFYYLFVVYLGPVSAAAIAATAVGFTGGLLARRFLIPPLIVAIAGITPMLPGLAIYRGMYATLNDQTLMGFTNIAVALATASSLAAGVVLGEWIARRLRRPPRFNPYRAFTKANEFSFQEEAEQNQRRQRKRPKTNQRFGNKR

SEQ ID NO: 11 - DNA, 44 bp, synthetic construct
  source 1..44: mol_type = other DNA
gtgtccgtgttcacctcagccgtatcttagcgtatgtgtacatc

SEQ ID NO: 12 - DNA, 43 bp, synthetic construct
  source 1..43: mol_type = other DNA
caaggtaccccgaccggtgcatttaagcgaaagacgcctaagc

SEQ ID NO: 13 - DNA, 624 bp, Corynebacterium glutamicum
  source 1..624: mol_type = genomic DNA
gtggacgcagcatcatgggtcgcattcgcactcgcattattggtggcattagcggtgcccggacctgaccttgttcttgttctacattctgcaacccgcgggatccgcacgggggtcatgactgcggcaggaatcatgacgggactgatgttacatgcgagtcttgcgatagccggagcaactgcattattgctatcagctccgggagtattgagcgctattcaacttcttggtgcgggagtgcttttgtggatgggcacgaacatgtttcgtgcttcccaaaataccggggaatctgaaactgctgctagtcaatcgagtgcaggttattttcgaggatttatcaccaatgccacgaacccgaaagcgctgttgttctttgcagcgattcttcctcagttcattgggaatggggaagatatgaaaatgaggaccttggcattgtgtgccaccatcgtgcttggctcaggagcgtggtggttgggaacaatcgcattggtcaggggtattggtctgcaaaagttaccgtctgcggatcgcattatcaccctggttggtggcatcgcactgtttctcattggtgccggattactggttaatactgcttatgggcttatcacttag

SEQ ID NO: 14 - protein, 207 aa, Corynebacterium glutamicum
VDAASWVAFALALLVALAVPGPDLVLVLHSATRGIRTGVMTAAGIMTGLMLHASLAIAGATALLLSAPGVLSAIQLLGAGVLLWMGTNMFRASQNTGESETAASQSSAGYFRGFITNATNPKALLFFAAILPQFIGNGEDMKMRTLALCATIVLGSGAWWLGTIALVRGIGLQKLPSADRIITLVGGIALFLIGAGLLVNTAYGLIT

SEQ ID NO: 15 - DNA, 40 bp, synthetic construct
  source 1..40: mol_type = other DNA
gtgtccgtgttcacctcagcgttcgaggattttccaatct

SEQ ID NO: 16 - DNA, 41 bp, synthetic construct
  source 1..41: mol_type = other DNA
caaggtaccccgaccggtgccctaagtgataagcccataag

SEQ ID NO: 17 - DNA, 44 bp, synthetic construct
  source 1..44: mol_type = other DNA
tcgagctcggtacccggggatcctctggctgatgatgatctcac

SEQ ID NO: 18 - DNA, 45 bp, synthetic construct
  source 1..45: mol_type = other DNA
ctagggatagtagacacttcacaagtgtcttacctcggctggttg

SEQ ID NO: 19 - DNA, 47 bp, synthetic construct
  source 1..47: mol_type = other DNA
cttgtgaagtgtctactatccctagaattactcagcgggcgcaccac

SEQ ID NO: 20 - DNA, 46 bp, synthetic construct
  source 1..46: mol_type = other DNA
agcttgcatgcctgcaggtcgactctgcacgacggttcagctcttc

(19) United States
(12) Patent Application Publication
(10) Pub. No.: US 2026/0275394 A1
(43) Pub. Date: Sep. 17, 2026

(54) IMPROVED BIOTECHNOLOGICAL PROCESS TO PRODUCE GUANIDINOACETIC ACID (GAA) BY TARGETED INTRODUCTION OR BY INCREASING THE ACTIVITY OF A TRANSMEMBRANE EXPORTER PROTEIN

(71) Applicant: Evonik Operations GmbH, Essen (DE)

(72) Inventors: Kay Marin, Borgholzhausen (DE); Frank Schneider, Halle (DE)

(73) Assignee: Evonik Operations GmbH, Essen (DE)

(21) Appl. No.: 19/124,954

(22) PCT Filed: Oct. 24, 2023
(86) PCT No.: PCT/EP2023/079587

§ 371 Date: Apr. 28, 2025

(30) Foreign Application Priority Data
Nov. 3, 2022 (EP) 22205232.6

(51) Int. Cl. C12P 13/00; C07K 14/34; C12N 9/10; C12N 15/52

(52) U.S. Cl. CPC C12P 13/005; C07K 14/34; C12N 9/1007; C12N 15/52; C12Y 201/01002; C12Y 201/04001; C12N 2830/34

(57) ABSTRACT

A microorganism has at least one gene coding for a protein having the function of a L-arginine:glycine amidinotransferase and having an increased activity of a transmembrane exporter protein. A method for the fermentative production of guanidino acetic acid (GAA) as well as a method for the fermentative production of creatine employ such microorganism.

Guanidino acetic acid (GAA) is a colorless crystalline organic compound used as animal feed additive (e.g. WO 2005120246 A1 and US 2011257075 A1). GAA is a natural precursor of creatine (e.g. Humm et al., Biochem. J. (1997) 322, 771-776). Therefore, the supplementation of GAA allows for an optimal supply of creatine in the organism.

The present invention pertains to a microorganism transformed to be capable of producing guanidinoacetic acid (GAA) and to a method for the fermentative production of GAA using such microorganism. The present invention also relates to a method for the fermentative production of creatine.

In biological systems GAA and ornithine are formed from arginine and glycine as starting materials by the catalytic action of an L-arginine:glycine-amidinotransferase (AGAT; EC 2.1.4.1). This reaction is also the first step in creatine biosynthesis.

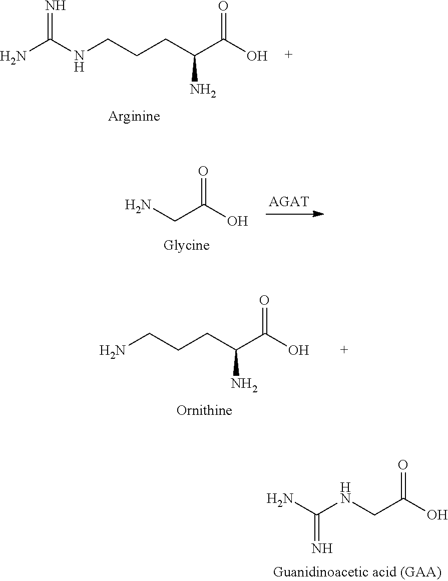 [SMILES: N=C(N)NCCC[C@H](N)C(=O)O ; NCC(=O)O ; NCCC[C@H](N)C(=O)O ; "+" ; N=C(N)NCC(=O)O]

Guthmiller et al. (J Biol Chem. 1994 Jul. 1; 269(26):17556-60) have characterized a rat kidney AGAT by cloning and heterologously expressing the enzyme in Escherichia coli (E. coli). Muenchhoff et al. (FEBS Journal 277 (2010) 3844-3860) report the first characterization of an AGAT from a prokaryote also by cloning and heterologously expressing the enzyme in E. coli.

Fan Wenchao discloses a method for the production of creatine by fermentation of non-pathogenic microorganisms, such as Corynebacterium glutamicum (CN106065411 A). The microorganism has the following biotransformation functions: glucose conversion to L-glutamic acid; conversion of L-glutamic acid to N-acetyl-L-glutamic acid; conversion of N-acetyl-L-glutamic acid to N-acetyl-L-glutamic acid semialdehyde; conversion of N-acetyl-L-glutamic acid semialdehyde to N-acetyl-L-ornithine; conversion of N-acetyl-L-ornithine to L-ornithine; conversion of L-ornithine to L-citrulline; conversion of L-citrulline to arginino-succinic acid; conversion of arginino-succinic acid to L-arginine; conversion of L-arginine to guanidinoacetic acid; and, finally, conversion of guanidinoacetic acid to creatine. Fan Wenchao proposes, that the microorganism overexpresses one or more enzymes selected from the group consisting of N-acetylglutamate-synthase, N-acetylornithine-5-aminotransferase, N-acetylornithinase, ornithine-carbamoyl transferase, argininosuccinate synthetase, glycine-amidinotransferase (L-arginine:glycine-amidinotransferase, EC: 2.1.4.1), and guanidinoacetate N-methyltransferase (EC: 2.1.1.2). The microorganism overexpresses preferably glycine-amidinotransferase (L-arginine:glycine amidinotransferase) and guanidinoacetate N-methyltransferase.

The production of guanidinoacetic acid (GAA) by a cyanobacterial cell expressing CyrA which encodes an L-arginine:glycine amidinotransferase (AGAT) as a first step in cylidrospermopsin biosynthesis and the involvement of CyrK as a putative cylindrospermopsin exporter is disclosed in WO2009129558 A1. The production of GAA in isolated Th17 cells from L-arginine and its export modulated by SAT1 is disclosed in WO2020191079 A1.

A microorganism capable of producing guanidinoacetic acid (GAA) was published by Zhang et al. (ACS Synth. Biol. 2020, 9, 2066-275). They designed a reconstituted ornithine cycle in E. coli by introducing a heterologous AGAT from different species (e.g., Homo sapiens, Cylindrospermopsis raciborskii, Moorena producens) and by introducing a citrulline synthesis module (e.g. ovexpression of argF and argi) and an arginine synthesis module (e.g. overexpression of argG, argH; introduction of aspA) into E. coli. Also CN111748506 A discloses the production of GAA by E. coli expressing AGAT whereas CN113487139 A discloses the production of GAA by Bacillus subtilis expressing GAA.

Schneider and Jankowitsch (WO 2021122400 A1) propose a method to produce GAA using a microorganism having a gene coding for a protein having the function of an L-arginine:glycine amidinotransferase (AGAT) and an increased carbamoyl phosphate synthase. The carbamoyl phosphate is an important precursor for the biosynthesis of GAA but also for L-arginine and other compounds.

To increase the production of GAA using a microorganism an intracellular high amount of the starting materials arginine and/or glycine is necessary. At the same time the byproduct of the AGAT reaction, L-ornithine, has to be recycled to L-arginine efficiently in order to prevent loss of carbon and energy. Several approaches for increasing the production of one of the starting materials in GAA synthesis, i.e. L-arginine, in microorganisms, particularly bacteria, are also known from literature. An overview for the metabolic engineering of Corynebacterium glutamicum (C. glutamicum) for L-arginine production is provided by Park et al. (NATURE COMMUNICATIONS|DOI: 10.1038/ncomms5618). Yim et al. (J Ind Microbiol Biotechnol (2011) 38:1911-1920) could show that inactivation of the argR, gene coding for the central repressor protein ArgR controlling the L-arginine biosynthetic pathway, in C. glutamicum leads to an improved arginine-producing strain. Ginesy et al. (Microbial Cell Factories (2015) 14:29) report the successful engineering of E. coli for enhanced arginine production. Among other, they proposed the deletion of the argR repressor gene. The deletion or inactivation of the argR repressor gene is also disclosed in connection with the production of GAA in WO2022008276 A1 and WO2022008280 A1.

On the other hand, there is also a need of an increased intracellular glycine concentration in order to increase the production of GAA. This is achieved by the internal synthesis of glycine as disclosed in WO2022008276 A1 and WO2022008280 A1. Alternatively, glycine can be added to the medium and membrane transport proteins facilitate the uptake.

If GAA production relies on the internal synthesis of arginine and glycine, the efficient interconnection of precursor supply (arginine and glycine) and product recycling (ornithine) as well export (GAA) is required. While ornithine recycling occurs via the reactions of the urea cycle, transport of GAA across cellular membranes in bacteria is not understood at all.

In general, chemical substances can cross biological membranes by passive diffusion or by active transport. For the transport of compounds that cannot diffuse through the membranes, transport proteins are responsible. These proteins permit active transport or facilitated diffusion of substances and are indispensable elements of living cells. Transport proteins can be specialized for the import of compounds, transport from the extracellular environment into the cytoplasm, or for the export of compounds, transport from the cytoplasm into the extracellular environment. Members of the first group are called importers or uptake systems, members of the second group are called exporters.

Transport proteins were classified in data bases like the “Transporter Classification Database” (Nucleic Acids Res (2006) 34: 181-6). The classification is based on sequence similarities and in the data base information on known substrates for the particular transport proteins is provided. Some transport proteins have a very limited and specific spectrum of substrates and others have a very broad substrate spectrum. In addition, transport proteins of different classes can transport the same compound. Despite the classification, the prediction of substrates that are transported by the particular transport protein based on its protein sequence is not possible yet.

It is important to notice that in contrast to globular soluble proteins the sequence similarity of membrane proteins (including transport proteins) is much less obvious. This depends on the fact that the major functional structural element of a membrane protein, the transmembrane helix, can be constituted by different amino acid compositions without changing its nature as transmembrane helix. As a consequence, the same protein functionalities can be observed within proteins sharing <20% sequence identity (Relation between sequence and structure in membrane proteins; M. Olivella, A. Gonzalez, L. Pardo and X. Deupi; Bioinformatics 2013 Vol. 29 Issue 13 Pages 1589-92; Accession Number: 23677941 DOI: 10.1093/bioinformatics/btt249).

For the optimization of the production of chemical compounds by bacteria for industrial application the efficient export of products out of the cell is key. For example, the lysine exporter LysE (encoded by lysE, i.e. NCgl1214) plays an important role for product excretion in strains of Corynebacterium glutamicum (J Bacteriol (1995) 177: 4021-7). On the one hand an accumulation of product inside the cell inhibits its own production by its impact on the chemical equilibrium of the final reaction or by the regulatory impact on the metabolic key reactions (Biosci Biotechnol Biochem (2001) 65:1149-54). On the other hand, accumulation of high concentrations of compounds inside the cell can have deleterious effects on growth and survival (Microbiology (2001) 147:1765-74).

The serE gene (=NCgl0580=cg0701=Cgl0605=CGL_RS03040; Seq ID NO:5) from Corynebacterium glutamicum (C. glutamicum) codes for a secondary transport protein of the “Drug/Metabolite Transporter (DMT) Superfamily” (TC 2.A.7; Saier M H Jr, Tran C V, Barabote R D. “TCDB: the Transporter Classification Database for membrane transport protein analyses and information.” Nucleic Acids Res. 2006 Jan. 1; 34 (Database issue): D181-6. doi: 10.1093/nar/gkj001. PMID: 16381841; PMCID: PMC1334385; RefSeq WP_011013759; Seq ID NO:6).

The SerE transport protein exports L-serine and L-threonine and its overexpression was found to improve the production of L-Serine (Zhang X, Gao Y, Chen Z, Xu G, Zhang X, Li H, Shi J, Koffas M A G, Xu Z. “High-yield production of L-serine through a novel identified exporter combined with synthetic pathway in Corynebacterium glutamicum.” Microb Cell Fact. 2020 May 29; 19(1):115. doi: 10.1186/s12934-020-01374-5. PMID: 32471433; PMCID: PMC7260847). SerE overexpression also improves L-cysteine production (Kishino M, Kondoh M, Hirasawa T. “Enhanced L-cysteine production by overexpressing potential L-cysteine exporter genes in an L-cysteine-producing recombinant strain of Corynebacterium glutamicum.” Biosci Biotechnol Biochem. 2019 December; 83(12):2390-2393. doi: 10.1080/09168451.2019.1659715. Epub 2019 Sep. 6. PMID: 31671040).

The thrE gene (=NCgl2533=cg2905=Cgl2622=CGL_RS13090; Seq ID NO:9) from C. glutamicum likely codes for a secondary transport protein of the “The Threonine/Serine Exporter (ThrE)” family (TC 2.A.79, Saier M H Jr, Tran C V, Barabote R D. “TCDB: the Transporter Classification Database for membrane transport protein analyses and information.” Nucleic Acids Res. 2006 Jan. 1; 34 (Database issue): D181-6. doi: 10.1093/nar/gkj001. PMID: 16381841; PMCID: PMC1334385; RefSeq WP_003853939; Seq ID NO:10). In the genome of C. glutamicum strain ATCC13032 it is annotated as a threonine/serine exporter family protein (RefSeq accession NC_003450, locus_tag=“CGL_RS13090”; see also Kalinowski J et al. 2003, J Biotechnol 104:5-25. 10.1016/S0168-1656(03)00154-8). Homologous proteins exist in selected bacteria, archaea and fungal eukaryotes (Yen M R, Tseng Y H, Simic P, Sahm H, Eggeling L, Saier M H Jr., Res Microbiol. 2002 January-February; 153(1):19-25. doi: 10.1016/s0923-2508(01)01281-5. PMID: 11881894).

ThrE exhibits 10 putative transmembrane α-helical spanners and it catalyses the proton motive force (pmf) dependent export of L-threonine and L-serine (Simic P, Sahm H, Eggeling L., J Bacteriol. 2001 September; 183(18):5317-24. doi: 10.1128/JB.183.18.5317-5324.2001. PMID: 11514515; PMCID: PMC95414; Simic et al., Appl Environ Microbiol. 2002; 68:3321-3327. doi: 10.1128/AEM.68.7.3321-3327.2002) and L-proline (Liu et al., Nat Commun. 2022 Feb. 16; 13(1):891. doi: 10.1038/s41467-022-28501-7. PMID: 35173152; PMCID: PMC8850433). The expression of thrE from C. glutamicum in an E. coli threonine producer significantly improved L-threonine production (Kruse et al., Appl Microbiol Biotechnol. 2002 July; 59(2-3):205-10. doi: 10.1007/s00253-002-0987-7. Epub 2002 Apr. 4. PMID: 12111147). Albeit having a similar function as the L-threonine exporters of Escherichia coli (RhtA, RhtB, and RhtC), a BLAST comparison shows no significant identity to those.

The gene NCgl2566 (=cg2941=Cgl2656=CGL_RS13250; Seq ID NO:13) from C. glutamicum codes likely for a secondary transport protein of the “LysE Superfamily” (RefSeq WP_011015285; Seq ID NO:14).

In the genome of C. glutamicum strain ATCC13032 it is annotated as a “LysE family translocator” (RefSeq accession NC_003450, locus_tag=“CGL_RS13250”). By a BLASTp search against the “Transporter Classification Database” (https://tcdb.org/progs/blast.php) and by the information on the web site the protein can be assigned as member of the LysE Superfamily accordingly (Tsu, Brian V.; Saier, Milton H. (2015) The LysE Superfamily of Transport Proteins Involved in Cell Physiology and Pathogenesis. PloS One 10 (10). doi: 10.1371/journal.pone.0137184. ISSN 1932-6203. PMC 4608589. PMID 26474485. More specifically, NCgl2566 can be assigned as a member of the RhtB family (TC #2.A.76) (cf. https://www.tcdb.org/search/result.php?tc=2.A.76).

Zakataeva (Zakataeva N P et al., Microb Cell Fact. 2020 May 29; 19(1):115. doi: 10.1186/s12934-020-01374-5. PMID: 32471433; PMCID: PMC7260847) has shown, that the deletion of rhtB in E. coli resulted in increased resistance to several amino acids, including L-threonine and L-serine. Overexpression of C. glutamicum NCgl2566 improves L-cysteine production (Kinisho et al., Biosci Biotechnol Biochem. 2019 December; 83(12):2390-2393. doi: 10.1080/09168451.2019.1659715. Epub 2019 Sep. 6. PMID: 31671040).

CN 108486133 A discloses a kind of L serine transport protein and its application. It has been found that in C. glutamicum NCgl0580 sequences have the function of an L serine transporter, named as serE. Its overexpression improves L-serine production in C. glutamicum.

WO 2021049866 A1 discloses a L-threonine export protein variant and a method for production of L-threonine using the same, in particular the expression of variants of the E. coli threonine exporter RhtC in C. glutamicum whereby the native thrE gene of C. glutamicum has been deleted.

WO 2012134253A2 concerns a Corynebacterium sp. transformed with a fructokinase gene derived from Escherichia sp. and a process for preparing L-amino acids using the same. In order to improve the export of L-threonine in C. glutamicum the expression of thrE has been increased.

The problem underlying the present inventions is to provide a microorganism transformed to be capable for producing guanidinoacetic acid (GAA) in higher amounts and a method for producing GAA using such microorganism.

The problem is solved by a microorganism comprising at least one heterologous or synthetic gene coding for a protein having the function of a L-arginine:glycine amidinotransferase (AGAT, e.g. EC2.1.4.1) and having an overexpressed gene coding for a transmembrane exporter protein wherein the transmembrane exporter protein is preferably selected from proteins of the Drug/Metabolite Transporter (DMT) superfamily (TC 2.A.7), from proteins of the 10 TMS Drug/Metabolite Exporter (DME) Family (TC 2.A.7.3), from proteins of the Threonine/Serine Exporter (ThrE) Family (TC 2.A.79), or from proteins of the Resistance to Homoserine/Threonine (RhtB) Family (TC 2.A.76).

A microorganism in the context of the present invention is an organism of microscopic size, which may exist in its single-celled form or as a colony of cells. Microorganisms include most unicellular organisms from all three domains of life. The domains Archaea and Bacteria contain microorganisms only. Among the third domain Eukaryota unicellular organism like yeasts, protists and protozoans belong to the group of microorganisms. Multicellular organisms or parts of them, like cell cultures, in particular cultures of animal cells, e.g. of human cells, are not considered as microorganism in the context of the present invention. Animal cells, e.g. human cells, are explicitly excluded from the definition of microorganisms in the context of the present invention.

The transmembrane exporter protein may be a transmembrane transport protein annotated as serine or threonine exporter.

Preferably, the transmembrane exporter protein has an identity of at least 20%, 30%, 40%, 50%, 60%, 70%, at least 80% or at least 90%, preferably is identical to a protein comprising the amino acid sequence according to SEQ ID NO:6 (SerE) or to a protein comprising the amino acid sequence according to SEQ ID NO:10 (ThrE) or to the protein comprising the amino acid sequence according to SEQ ID NO:14.

In the present context the extent to which protein sequences are related is expressed as identity meaning more specifically, how much of the sequence is identical according to the results of a blast search given as percentage (see https://www.ncbi.nlm.nih.gov/books/NBK62051/ for definition).

In a further embodiment of the present invention the transmembrane exporter protein is encoded by the open reading frame of the NCgl0580 gene of Corynebacterium glutamicum (i.e. the serE gene=cg0701=Cgl0605=CGL_RS03040 from C. glutamicum) according to SEQ ID NO: 5.

In a further embodiment of the present invention the transmembrane exporter protein is encoded by the open reading frame of the NCgl2533 (thrE) gene of Corynebacterium glutamicum according to SEQ ID NO: 9.

In a further embodiment of the present invention the transmembrane exporter protein is encoded by the open reading frame of the NCgl2566 gene of Corynebacterium glutamicum according to SEQ ID NO:13.

The microorganism according to the present invention is preferably a genetically modified organism that does not naturally occur. In such a microorganism the genetic material has been altered using genetic engineering techniques. According to the present invention, the open reading frame of the gene coding for a protein having the function of an L-arginine:glycine amidinotransferase (AGAT, e.g. EC 2.1.4.1) is heterologous or synthetic, meaning that at least one gene coding for a protein having the function of an L-arginine:glycine amidinotransferase has been introduced using genetic engineering techniques.

A heterologous or synthetic gene means that the gene has been inserted into a host organism which does not naturally have this gene. Insertion of the heterologous or synthetic gene in the host is performed by recombinant DNA technology. Microorganisms that have undergone recombinant DNA technology are called transgenic, genetically modified or recombinant. A heterologous protein means a protein that is not naturally occurring in the microorganism. A homologous or endogenous gene means that the gene including its function as such, or the nucleotide sequence of the gene is naturally occurring in the microorganism or is “native” in the microorganism. A homologous or a native protein means a protein that is naturally occurring in the microorganism.

In the microorganism according to the present invention the activity of the protein having the function of an L-arginine:glycine amidinotransferase (AGAT) is preferably increased compared to the respective activity in the wildtype organism by overexpression of the gene coding for the protein having the function of an L-arginine:glycine amidinotransferase.

In the microorganism according to the present invention the protein having the function of an L-arginine:glycine amidinotransferase (AGAT) comprises an amino acid sequence which is at least 80% identical to the amino acid sequence according to SEQ ID NO: 2.

Increased enzyme activities in a microorganism usually are achieved by overexpressing the genes coding for the respective enzymes.

Overexpression of a gene is generally achieved by increasing the copy number of the gene and/or by functionally linking the gene with a strong promoter and/or by enhancing the ribosomal binding site and/or by codon usage optimization of the start codon or of the whole gene and/or by modifying activities of proteins modulating gene expression or a combination comprising a selection of all methods mentioned above.

In the microorganism of the present invention the overexpression of the gene coding for a transmembrane exporter protein may be achieved by increasing the copy number of the gene and/or by functionally linking the gene with a strong promoter and/or by enhancing the ribosomal binding site and/or by codon usage optimization of the start codon or of the whole gene and/or by modifying activities of proteins modulating gene expression.

A promoter is a DNA sequence consisting of about 40 to 50 base pairs and which constitutes the binding site for an RNA polymerase holoenzyme and the transcriptional start point, whereby the strength of expression of the controlled polynucleotide or gene can be influenced. Generally, it is possible to achieve overexpression or an increase in the expression of genes in bacteria by the use of strong promoters, for example by replacing original promoters with strong promoters or by placing additional strong promoters upstream of the gene of interest. Such strong promoter sequences can be native (e.g., originally assigned to other sequences of the same species), they can be heterologous (e.g., derived from the DNA of other species) or they can be partially or fully have a synthetic origin. Overexpression can also be achieved by modifying certain regions of a given, native promoter (for example its so-called −10 and −35 regions), e.g. as taught by M. Patek et al. (Microbial Biotechnology 6 (2013), 103-117) for C. glutamicum. The microorganism according to the present invention may have an increased ability to produce L-arginine from L-ornithine compared with the ability of the wildtype microorganism.

In the context of the present invention, a microorganism having an increased ability to produce L-arginine means a microorganism producing L-arginine in excess of its own need. Examples for such L-arginine producing microorganisms are e.g. C. glutamicum ATCC 21831 or those disclosed by Park et al. (NATURE COMMUNICATIONS|DOI: 10.1038/ncomms5618) or by Ginesy et al. (Microbial Cell Factories (2015) 14:29). In contrast to formerly described microorganisms having an increased ability to produce L-arginine, L-arginine excretion is not desirable in strains for GAA production since arginine is utilized inside the cell in the framework of the present invention for GAA production.

In a further embodiment the microorganism according to the present invention the expression of an argR gene coding for the arginine responsive repressor protein ArgR is attenuated compared to the expression of the argR gene in the wildtype microorganism. Alternatively, the argR gene is inactivated or deleted.

The microorganism of the present invention may belong to the genus Corynebacterium, to the genus Bacillus (Yan, K., et al. (2022). “Biosynthesis of Guanidinoacetate by Bacillus subtilis Whole-Cell Catalysis.” Fermentation 8(3):116), to the genus Enterobacteriaceae or to the genus Pseudomonas.

In a particular embodiment of the present invention the microorganism is Corynebacterium glutamicum (C. glutamicum) or Escherichia coli (E. coli).

The present invention further concerns a method for the fermentative production of guanidino acetic acid (GAA), comprising the steps of cultivating the microorganism according to the present invention in a medium, and accumulating GAA in the medium to form a GAA containing fermentation broth.

Preferably, the method further comprises isolating GAA from the GAA containing fermentation broth.

In a particular embodiment the microorganism of the present invention further comprises a gene coding for an enzyme having the activity of a guanidinoacetate N-methyltransferase. The gene coding for an enzyme having the activity of a guanidinoacetate N-methyltransferase may be overexpressed.

The present invention also concerns a method for the fermentative production of creatine, comprising the steps of a) cultivating the microorganism according to the present invention further comprising a gene coding for an enzyme having the activity of a guanidinoacetate N-methyltransferase in a suitable medium under suitable conditions, and b) accumulating creatine in the medium to form a creatine containing fermentation broth.

Preferably, the method further comprises isolating creatine from the creatine containing fermentation broth. Creatine may be extracted from fermentation broth by isoelectric point method and/or ion exchange method. Alternatively, creatine can be further purified by a method of recrystallization in water.

### BRIEF DESCRIPTION OF THE SEQUENCES

SEQ ID NO:1: Moorena producens strain PAL-8-15-08-1 DNA sequence of locus_tag BJP34_00300 coding for a L-arginine:glycine amidinotransferase (AGAT, EC 2.1.4.1)
SEQ ID NO:2: Derived amino acid sequence from locus_tag BJP34_00300 (RefSeq accession Number WP_070390602)
SEQ ID NO:3: Synthetic DNA fragment for cloning a plasmid for the expression of the L-arginine:glycine amidinotransferase of Moorena producens strain PAL-8-15-08-1
SEQ ID NO:4: DNA Sequence of the shuttle plasmid pLIB_P, used for gene expression in C. glutamicum
SEQ ID NO:5: DNA sequence of the open reading frame serE (NCgl0580=CGL_RS03040) of C. glutamicum strain ATCC13032
SEQ ID NO:6: Derived amino acid sequence from the open reading frame serE of C. glutamicum strain ATCC13032 (RefSeq accession Number WP_011013759)
SEQ ID NO:7: PCR primer serE_f_NotI used for amplification of the ORF serE
SEQ ID NO:8: PCR primer serE_r_NotI used for amplification of the ORF serE
SEQ ID NO:9: DNA sequence of the open reading frame thrE (NCgl2533=CGL RS13090) of C. glutamicum strain ATCC13032
SEQ ID NO:10: Derived amino acid sequence from the open reading frame thrE of C. glutamicum strain ATCC13032 (RefSeq accession Number WP_003853939)
SEQ ID NO:11: PCR primer thrE_f_NotI used for amplification of the ORF thrE
SEQ ID NO:12: PCR primer thrE_r_NotI used for amplification of the ORF thrE
SEQ ID NO:13: DNA sequence of the open reading frame NCgl2566 (CGL_RS13250) of C. glutamicum strain ATCC13032
SEQ ID NO:14: Derived amino acid sequence from the open reading frame NCgl2566 of C. glutamicum strain ATCC13032 (RefSeq accession Number RefSeq WP_011015285)
SEQ ID NO:15: PCR primer NCgl2566_f_NotI used for amplification of the ORF NCgl2566
SEQ ID NO:16: PCR primer NCgl2566_r_NotI used for amplification of the ORF NCgl2566
SEQ ID NO:17: PCR primer DargR_lf used for amplification of the left homology arm of the argR deletion plasmid pK18mobsacB_DargR
SEQ ID NO:18: PCR primer DargR_lr used for amplification of the left homology arm of the argR deletion plasmid pK18mobsacB_DargR
SEQ ID NO:19: PCR primer DargR_rf used for amplification of the right homology arm of the argR deletion plasmid pK18mobsacB_DargR
SEQ ID NO:20: PCR primer DargR_rr used for amplification of the right homology arm of the argR deletion plasmid pK18mobsacB_DargR

### A) MATERIALS and METHODS

### Chemicals

Kanamycin solution from Streptomyces kanamyceticus was purchased from Sigma Aldrich (St. Louis, USA, Cat. no. K0254). If not stated otherwise, all other chemicals were purchased analytically pure from Merck (Darmstadt, Germany), Sigma Aldrich (St. Louis, USA) or Carl-Roth (Karlsruhe, Germany).

### Cultivation for Cell Proliferation

If not stated otherwise, cultivation/incubation procedures were performed as follows herewith:
a. LB broth (MILLER) from Merck (Darmstadt, Germany; Cat. no. 110285) was used to cultivate E. coli strains in liquid medium. The liquid cultures (10 ml liquid medium per 100 ml Erlenmeyer flask with 3 baffles) were incubated in the Infors HT Multitron standard incubator shaker from Infors GmbH (Bottmingen, Switzerland) at 30° C. and 200 rpm.
b. LB agar (MILLER) from Merck (Darmstadt, Germany, Cat. no. 110283) was used for cultivation of E. coli strains on agar plates. The agar plates were incubated at 30° C. in an INCU-Line® mini incubator from VWR (Radnor, USA).
c. Brain heart infusion broth (BHI) from Merck (Darmstadt, Germany, Cat. no. 110493) was used to cultivate C. glutamicum strains in liquid medium. The liquid cultures (10 ml liquid medium per 100 ml Erlenmeyer flask with 3 baffles) were incubated in the Infors HT Multitron standard incubator shaker from Infors GmbH (Bottmingen, Switzerland) at 30° C. and 200 rpm.
d. Brain heart agar (BHI-agar) from Merck (Darmstadt, Germany, Cat. no. 113825) was used for cultivation of C. glutamicum strains on agar plates. The agar plates were incubated at 30° C. in an incubator from Heraeus Instruments with Kelvitron® temperature controller (Hanau, Germany).
e. For cultivating C. glutamicum after electroporation, BHI-agar (Merck, Darmstadt, Germany, Cat. no. 113825) was supplemented with 134 g/I sorbitol (Carl Roth GmbH+Co. KG, Karlsruhe, Germany), 2.5 g/I yeast extract (Oxoid/ThermoFisher Scientific, Waltham, USA, Cat. no. LP0021) and 25 mg/l kanamycin. The agar plates were incubated at 30° C. in an incubator from Heraeus Instruments with Kelvitron® temperature controller (Hanau, Germany).

### Determining Optical Density of Bacterial Suspensions

a. The optical density of bacterial suspensions in shake flask cultures was determined at 600 nm (OD600) using the Bio-Photometer from Eppendorf AG (Hamburg, Germany).
b. The optical density of bacterial suspensions produced in the Wouter Duetz (WDS) micro fermentation system (24-Well Plates) was determined at 660 nm (OD660) with the GENios™ plate reader from Tecan Group AG (Mannedorf, Switzerland).

### Centrifugation

a. Bacterial suspensions with a maximum volume of 2 ml were centrifuged in 1.5 ml or 2 ml reaction tubes (e.g. Eppendorf Tubes® 3810X) using an Eppendorf 5417 R benchtop centrifuge (5 min. at 13.000 rpm).
b. Bacterial suspensions with a maximum volume of 50 ml were centrifuged in 15 ml or 50 ml centrifuge tubes (e.g. Falcon™ 50 ml Conical Centrifuge Tubes) using an Eppendorf 5810 R benchtop centrifuge for 10 min. at 4.000 rpm.

### DNA Isolation

Plasmid DNA was isolated from E. coli cells using the QIAprep Spin Miniprep Kit from Qiagen (Hilden, Germany, Cat. No. 27106) according to the instructions of the manufacturer.

### Polymerase Chain Reaction (PCR)

PCR with a proof reading (high fidelity) polymerase was used to amplify a desired segment of DNA for sequencing or DNA assembly. Non-proof-reading polymerase Kits were used for determining the presence or absence of a desired DNA fragment directly from E. coli or C. glutamicum colonies.
a. The Phusion® High-Fidelity DNA Polymerase Kit (Phusion Kit) from New England BioLabs Inc. (Ipswich, USA, Cat. No. M0530) was used for template-correct amplification of selected DNA regions according to the instructions of the manufacturer (see Table 1).

TABLE 1
Thermocycling conditions for PCR with Phusion ® High-
Fidelity DNA Polymerase Kit from New England BioLabs Inc.
PCR Program

| | | T | |
|---|---|---|---|
| Step | Time [min.:sec.] | [° C.] | Description |
| 1 | 00:30 | 98 | Initial denaturation step |
| 2 | 00:05 | 98 | Denaturation step |
| 3 | 00:30 | 60 | Annealing step |
| 4 | 30 sec. per kb DNA | 72 | Elongation step |
| | | | Repeat step 2 to 4: 35× |
| 5 | 05:00 | 72 | Final elongation step |
| 6 | Hold | 4 | Cooling step |

b. Taq PCR Core Kit (Taq Kit) from Qiagen (Hilden, Germany, Cat. No. 201203) was used to amplify a desired segment of DNA to confirm its presence. The kit was used according to the instructions of the manufacturer (see Table 2).

TABLE 2
Thermocycling conditions for PCR with Taq
PCR Core Kit (Taq Kit) from Qiagen.
PCR Program

| | Time | T | |
|---|---|---|---|
| Step | [min.:sec.] | [° C.] | Description |
| 1 | 05:00 | 94 | Initial denaturation step |
| 2 | 00:30 | 94 | Denaturation step |
| 3 | 00:30 | 52 | Annealing step |
| 4 | 1 min. per kb DNA | 72 | Elongation step |
| | | | Repeat step 2 to 4: 35× |
| 5 | 04:00 | 72 | Final elongation step |
| 6 | Hold | 4 | Cooling step |

c. SapphireAmp® Fast PCR Master Mix (Sapphire Mix) from Takara Bio Inc (Takara Bio Europe S.A.S., Saint-Germain-en-Laye, France, Cat. No. RR350A/B) was used as an alternative to confirm the presence of a desired segment of DNA in cells taken from E. coli or C. glutamicum colonies according to the instructions of the manufacturer (see Table 3).

TABLE 3
Thermocycling conditions for PCR with SapphireAmp ®
Fast PCR Master Mix (Sapphire Mix) from Takara Bio Inc.
PCR Program

| | | T | |
|---|---|---|---|
| Step | Time [min.:sec.] | [° C.] | Description |
| 1 | 01:00 | 94 | Initial denaturation step |
| 2 | 00:05 | 98 | Denaturation step |
| 3 | 00:05 | 55 | Annealing step |
| 4 | 10 sec. per kb DNA | 72 | Elongation step |
| | | | Repeat step 2 to 4: 30× |
| 5 | 04:00 | 72 | Final elongation step |
| 6 | Hold | 4 | Cooling step |

d. All oligonucleotide primers were synthesized by Eurofins Genomics GmbH (Ebersberg, Germany).
e. As PCR template either a suitably diluted solution of isolated plasmid DNA or of total DNA isolated from a liquid culture or the total DNA contained in a bacterial colony (colony PCR) was used. For said colony PCR the template was prepared by taking cell material with a sterile toothpick from a colony on an agar plate and placing the cell material directly into the PCR reaction tube. The cell material was heated for 10 sec. with 800 W in a microwave oven type Mikrowave & Grill from SEVERIN Elektrogerste GmbH (Sundern, Germany) and then the PCR reagents were added to the template in the PCR reaction tube.
f. All PCR reactions were carried out in PCR cyclers type Mastercycler or Mastercycler nexus gradient from Eppendorf AG (Hamburg, Germany).

### Restriction Enzyme Digestion of DNA

For restriction enzyme digestions either “FastDigest restriction endonucleases (FD)” (ThermoFisher Scientific, Waltham, USA) or restriction endonucleases from New England BioLabs Inc. (Ipswich, USA) were used. The reactions were carried out according to the instructions of the manufacturer's manual.

### Determining the Sizes of DNA Fragments

a. The sizes of small DNA fragments (<1000 bps) were usually determined by automatic capillary electrophoresis using the QIAxcel from Qiagen (Hilden, Germany).
b. If DNA fragments needed to be isolated or if the DNA fragments were >1000 bps DNA was separated by TAE agarose gel electrophoresis and stained with GelRed® Nucleic Acid Gel Stain (Biotium, Inc., Fremont, Canada). Stained DNA was visualized at 302 nm.

### Purification of PCR Amplificates and Restriction Fragments

PCR amplificates and restriction fragments were cleaned up using the QIAquick PCR Purification Kit from Qiagen (Hilden, Germany; Cat. No. 28106), according to the manufacturer's instructions. DNA was eluted with 30 μl 10 mM Tris*HCl (pH 8.5).

### Determining DNA Concentration

DNA concentration was measured using the NanoDrop Spectrophotometer ND-1000 from PEQLAB Biotechnologie GmbH, since 2015 VWR brand (Erlangen, Germany).

### Assembly Cloning

Plasmid vectors were assembled using the “NEBuilder HiFi DNA Assembly Cloning Kit” purchased from New England BioLabs Inc. (Ipswich, USA, Cat. No. E5520). The reaction mix, containing the linear vector and at least one DNA insert, was incubated at 50° C. for 60 min. 0.5 μl of Assembly mixture was used for each transformation experiment.

Chemical Transformation of E. coli

For plasmid cloning, chemically competent “NEB® Stable Competent E. coli (High Efficiency)” (New England BioLabs Inc., Ipswich, USA, Cat. No. C3040) were transformed according to the manufacturer's protocol. Successfully transformed cells were selected on LB agar supplemented with 25 mg/l kanamycin.

Transformation of C. glutamicum

Transformation of C. glutamicum with plasmid-DNA was conducted via electroporation using a “Gene Pulser Xcell” (Bio-Rad Laboratories GmbH, Feldkirchen, Germany) as described by Ruan et al. (2015). Electroporation was performed in 1 mm electroporation cuvettes (Bio-Rad Laboratories GmbH, Feldkirchen, Germany) at 1.8 kV and a fixed time constant set to 5 ms. Transformed cells were selected on BHI-agar containing 134 g/I sorbitol, 2.5 g/I Yeast Extract and 25 mg/l kanamycin.

C. glutamicum Strains

Corynebacterium glutamicum ATCC13032 (DSM 20300, Kinoshita S, Udaka S, Shimono M., J. Gen. Appl. Microbiol. 1957; 3(3): 193-205), the C. glutamicum wild type strain, is commercially available at the American Type Culture Collection (ATCC) or at the DSMZ-German Collection of Microorganisms and Cell Cultures GmbH.

### Determining Nucleotide Sequences

Nucleotide sequences of DNA molecules were determined by Eurofins Genomics GmbH (Ebersberg, Germany) by cycle sequencing, using the dideoxy chain termination method of Sanger et al. (Proceedings of the National Academy of Sciences USA 74, 5463-5467, 1977). Clonemanager Professional 9 software from Scientific & Educational Software (Denver, USA) was used to visualize and evaluate the sequences as well as for in silico assembly of sequences.

Glycerol Stocks of E. coli and C. glutamicum Strains

For long time storage of E. coli- and C. glutamicum strains glycerol stocks were prepared. Selected E. coli clones were cultivated in 10 ml LB medium supplemented with 2 g/I glucose. Selected C. glutamicum clones were cultivated in 10 ml twofold concentrated BHI medium supplemented with 2 g/I glucose. Media for growing plasmid containing E. coli- and C. glutamicum strains were supplemented with 25 mg/l kanamycin. The medium was contained in 100 ml Erlenmeyer flasks with 3 baffles. It was inoculated with a loop of cells taken from a colony. The culture was then incubated for 18 h at 30° C. and 200 rpm. After said incubation period 1.2 ml 85% (v/v) sterile glycerol were added to the culture. The obtained glycerol containing cell suspension was then aliquoted in 2 ml portions and stored at −80° C.

### GAA Production in Small-Scale Cultivations

The millilitre-scale cultivation system according to Duetz (2007) was used to assess the GAA-production of the strains. For this purpose, 24-deepwell microplates (24 well WDS plates) from EnzyScreen BV (Heemstede, Netherlands, Cat. no. CR1424) filled with 2.5 ml medium per well were used.

Precultures of the strains were done in 10 ml seed medium (SM). The medium was contained in a 100 ml Erlenmeyer flask with 3 baffles. It was inoculated with 100 μl of a glycerol stock culture and the culture was incubated for 24 h at 30° C. and 200 rpm. The composition of the seed medium (SM) is shown in Table 4.

TABLE 4
Seed medium (SM)

| Components | Concentration (g/l) |
|---|---|
| Yeast extract FM902 (Angel Yeast | 10 |
| Co., LTD, Hubei, P.R. China) | |
| Urea | 1.5 |
| KH₂PO₄ | 0.5 |
| K₂HPO₄ | 0.5 |
| MgSO₄ * 7H₂O | 1 |
| Biotin | 0.0001 |
| Thiamine hydrochloride | 0.0001 |
| FeSO₄ * 7H₂O | 0.01 |
| MnSO₄ * H₂O | 0.01 |
| Glucose | 20 |
| Kanamycin | 0.025 |
| pH = 7.0 | |

After said incubation period the optical densities OD600 of the precultures were determined. The volume, needed to inoculate 2.5 ml of production medium (PM) to an OD600 of 0.1, was sampled from the preculture, centrifuged (1 m at 8000 g) and the supernatant was discarded. Cells were then resuspended in 100 μl of production medium.

The main cultures were started by inoculating the 2.4 ml production medium (PM) containing wells of the 24 Well WDS-Plate with each 100 μl of the resuspended cells from the precultures. The composition of the production medium (PM) is shown in Table 5.

TABLE 5
Production medium (PM)

| Components | Concentration (g/l) |
|---|---|
| 3-(N-morpholino)propanesulfonic acid (MOPS) | 40 |
| Yeast extract FM902 (Angel Yeast Co., | 1.5 |
| LTD, Hubei, P.R. China) | |
| (NH₄)₂SO₄ | 10 |
| NH₄Cl | 15 |
| Trisodium citrate * 2H₂O | 10 |
| Urea | 1 |
| KH₂PO₄ | 0.5 |
| K₂HPO₄ | 0.5 |
| Ammonium acetate | 7.7 |
| MgSO₄ * 7H₂O | 1 |
| Biotin | 0.0001 |
| Thiamine hydrochloride | 0.0001 |
| FeSO₄ * 7H₂O | 0.01 |
| MnSO₄ * H₂O | 0.01 |
| ZnSO₄ * 7H₂O | 0.00002 |
| CuSO₄ * 5H₂O | 0.0004 |
| Antifoam XFO-1501 (Ivanhoe | 0.5 |
| Industries Inc., Zion, USA) | |
| Glucose | 40 |
| L-Arginine | 1.9 |
| Glycin | 0.818 |
| Kanamycin | 0.025 |
| pH = 7.2 | |

The main cultures were incubated for 72 h at 30° C. and 225 rpm in an Infors HT Multitron standard incubator shaker from Infors GmbH (Bottmingen, Switzerland) until complete consumption of glucose. The glucose concentration in the suspension was analyzed with the blood glucose-meter OneTouch Vita® from LifeScan (Johnson & Johnson Medical GmbH, Neuss, Germany).

After cultivation the culture suspensions were transferred to a deep well microplate. A part of the culture suspension was suitably diluted to measure the OD600. Another part of the culture was centrifuged and the concentration of GAA in the supernatant was analyzed as described below.

### Quantification of GAA

Samples were analyzed with an analyzing system from Agilent, consisting of a HPLC “Infinity 1260” coupled with a mass analyzer “Triple Quad 6420” (Agilent Technologies Inc., Santa Clara, USA). Chromatographic separation was done on the Atlantis HILIC Silica column, 4.6×250 mm, 5 μm (Waters Corporation, Milford, USA) at 35° C. Mobile phase A was water with 10 mM ammonium formate and 0.2% formic acid. Mobile phase B was a mixture of 90% acetonitrile and 10% water, 10 mM ammonium formate were added to the mixture. The HPLC system was started with 100% B, followed by a linear gradient for 22 min and a constant flow rate of 0.6 mL/min to 66% B. The mass analyzer was operated in the ESI positive ionization mode. For detection of GAA the m/z values were monitored by using an MRM fragmentation [M+H]+118-76. The limit of quantification (LOQ) for GAA was fixed to 7 ppm.

### B) EXPERIMENTAL RESULTS

### Example 1: Cloning of the Gene AGAT-Mp Coding for an L-Arginine:Glycine Amidinotransferase (AGAT, EC 2.1.4.1) from Moorena producens

Moorena producens is a filamentous cyanobacterium. The genome of the Moorena producens strain PAL-8-15-08-1 was published by Leao et al. (Leao T, Castelao G, Korobeynikov A, Monroe E A, Podell S, Glukhov E, Allen E E, Gerwick W H, Gerwick L, Proc Natl Acad Sci USA. 2017 Mar. 21; 114(12):3198-3203. doi: 10.1073/pnas.1618556114; RefSeq accession Number CP017599). It contains an open reading frame coding for a L-arginine:glycine amidinotransferase (AGAT, EC 2.1.4.1; locus_tag BJP34_00300 shown in SEQ ID NO:1). SEQ ID NO:2 shows the derived amino acid sequence (RefSeq accession Number WP_070390602).

Using the software tool “Optimizer” (http://genomes.urv.es/OPTIMIZER/) the amino acid sequence was translated back into a DNA sequence optimized for the codon usage of C. glutamicum. The 5′-end of the optimized gene was expanded with a BsaI restriction site, a 5′-UTR sequence for assembly cloning and a ribosomal binding site. At the 3′-end a second stop-codon, a sequence for assembly cloning and a BsaI-site was added. The resulting DNA sequence was named AGAT-Mp-insert (SEQ ID NO:3), it was ordered for gene synthesis from Eurofins Genomics GmbH (Ebersberg, Germany) and it was delivered as part of a cloning plasmid with an ampicillin resistance gene (designated as pEX-A258_AGAT-Mp).

The E. coli-C. glutamicum shuttle plasmid pLIB_P consists of the replication origin from pBL1 (for C. glutamicum), the pSC101 replication origin (for E. coli) and a kanamycin resistance gene. Following a unique NotI restriction site it has a strong promoter, two inversely orientated BsaI-sites and the BioBricks Terminator BBa_B1006 (SEQ ID NO:4).

pLIB_P was digested using the restriction endonuclease BsaI and the DNA was purified with the “QIAquick PCR Purification Kit” (Qiagen GmbH, Hilden, Germany).

The cloning plasmid pEX-A258_AGAT-Mp was digested using the restriction endonuclease BsaI and the DNA was purified with the “QIAquick PCR Purification Kit” (Qiagen GmbH, Hilden, Germany).

The DNA solutions of BsaI digested pLIB_P and pEX-A258_AGAT-Mp were joined, and matching sequence ends were assembled using the “NEBuilder HiFi DNA Assembly Cloning Kit” (New England BioLabs Inc., Ipswich, USA, Cat. No. E5520). The product was transformed into “NEB Stable Competent E. coli (High Efficiency)” (New England Biolabs, Ipswich, USA) and cells were grown on LB agar containing 25 mg/l kanamycin. Proper plasmid clones were identified by restriction digestion and DNA sequencing. The resulting plasmid was named pLIB_P_AGAT-Mp.

### Example 2: Cloning of the Expression Plasmid pLIB_serE_AGAT-Mp Coding for SerE Protein (NCgl0580) from Corynebacterium glutamicum and the L-Arginine:Glycine Amidinotransferase (AGAT, EC 2.1.4.1) from Moorena producens

The expression plasmid pLIB_serE_AGAT-Mp contains the serE gene (NCgl0580, Seq ID NO:5) from Corynebacterium glutamicum positioned upstream of the AGAT gene and its promoter.

A PCR was conducted using the primers serE_f NotI (Seq ID NO:7) and serE_r_NotI (Seq ID NO:8) and genomic DNA of Corynebacterium glutamicum ATCC13032 as a template. The resulting PCR product contained the serE gene (NCgl0580) plus 41 bp sequence upstream of the atg start codon. It was purified with the “QIAquick PCR Purification Kit” (Qiagen GmbH, Hilden, Germany).

The Plasmid pLIB_P_AGAT-Mp was digested using the restriction endonuclease NotI and the DNA was purified with the “QIAquick PCR Purification Kit” (Qiagen GmbH, Hilden, Germany).

The DNA of NotI-digested pLIB_P_AGAT-Mp was joined with the serE containing PCR product and the matching sequence ends were assembled using the “NEBuilder HiFi DNA Assembly Cloning Kit” (New England BioLabs Inc., Ipswich, USA, Cat. No. E5520).

The product was transformed into “NEB Stable Competent E. coli (High Efficiency)” (New England Biolabs, Ipswich, USA) and cells were grown on LB agar containing 25 mg/l kanamycin. Proper plasmid clones were identified by restriction digestion and DNA sequencing. The resulting plasmid was named pLIB_serE_AGAT-Mp.

### Example 3: Cloning of the Expression Plasmid pLIB_thrE_AGAT-Mp Coding for ThrE Protein (NCgl2533) from Corynebacterium glutamicum and the L-Arginine:Glycine Amidinotransferase (AGAT, EC 2.1.4.1) from Moorena producens

The expression plasmid pLIB_thrE_AGAT-Mp contains the thrE gene (NCgl2533, Seq ID NO:9) from Corynebacterium glutamicum positioned upstream of the AGAT gene and its promoter.

A PCR was conducted using the primers thrE_f_NotI (Seq ID NO:11) and thrE_r_NotI (Seq ID NO:12) and genomic DNA of Corynebacterium glutamicum ATCC13032 as a template. The resulting PCR product contained the thrE gene (NCgl2533) plus 114 bp sequence upstream of the atg start codon. It was purified with the “QIAquick PCR Purification Kit” (Qiagen GmbH, Hilden, Germany).

The Plasmid pLIB_P_AGAT-Mp was digested using the restriction endonuclease NotI and the DNA was purified with the “QIAquick PCR Purification Kit” (Qiagen GmbH, Hilden, Germany).

The DNA of NotI-digested pLIB_P_AGAT-Mp was joined with the thrE containing PCR product and the matching sequence ends were assembled using the “NEBuilder HiFi DNA Assembly Cloning Kit” (New England BioLabs Inc., Ipswich, USA, Cat. No. E5520).

The product was transformed into “NEB Stable Competent E. coli (High Efficiency)” (New England Biolabs, Ipswich, USA) and cells were grown on LB agar containing 25 mg/l kanamycin. Proper plasmid clones were identified by restriction digestion and DNA sequencing. The resulting plasmid was named pLIB_thrE_AGAT-Mp.

### Example 4: Cloning of the Expression Plasmid pLIB_NCgl2566_AGAT-Mp Coding for the NCgl2566 Protein from Corynebacterium glutamicum and the L-Arginine:Glycine Amidinotransferase (AGAT, EC 2.1.4.1) from Moorena producens

The expression plasmid pLIB_NCgl2566_AGAT-Mp contains the gene NCgl2566 (Seq ID NO:13) from Corynebacterium glutamicum positioned upstream of the AGAT gene and its promoter.

A PCR was conducted using the primers NCgl2566_f_NotI (Seq ID NO:15) and NCgl2566_r_NotI (Seq ID NO:16) and genomic DNA of Corynebacterium glutamicum ATCC13032 as a template. The resulting PCR product contained the gene NCgl2566 plus 141 bp sequence upstream of the gtg start codon. It was purified with the “QIAquick PCR Purification Kit” (Qiagen GmbH, Hilden, Germany).

The Plasmid pLIB_P_AGAT-Mp was digested using the restriction endonuclease NotI and the DNA was purified with the “QIAquick PCR Purification Kit” (Qiagen GmbH, Hilden, Germany).

The DNA of NotI-digested pLIB_P_AGAT-Mp was joined with the NCgl2566 containing PCR product and the matching sequence ends were assembled using the “NEBuilder HiFi DNA Assembly Cloning Kit” (New England BioLabs Inc., Ipswich, USA, Cat. No. E5520).

The product was transformed into “NEB Stable Competent E. coli (High Efficiency)” (New England Biolabs, Ipswich, USA) and cells were grown on LB agar containing 25 mg/l kanamycin. Proper plasmid clones were identified by restriction digestion and DNA sequencing. The resulting plasmid was named pLIB_NCgl2566_AGAT-Mp.

### Example 5: Chromosomal Deletion of the Gene argR (NCgl1345) in ATCC13032

To improve intracellular L-Arginine formation and L-Arginine recycling from L-Ornithine, the gene argR (NCgl1345) coding for the central repressor protein ArgR controlling the L-arginine biosynthetic pathway was inactivated.

Therefore, the plasmid pK18mobsacB_DargR was constructed as follows. Plasmid pK18mobsacB (Schafer, 1994) was cut using XbaI and the linearized vector DNA (5721 bps) was purified using the “QIAquick Gel Extraction Kit” (Qiagen GmbH, Hilden, Germany).

For constructing the insert, two DNA fragments were created by PCR with the following pairs of primers (genomic DNA of ATCC13032 as template):

$$DargR\_If ( SEQ ID NO : 17 ) , \text{} + DargR\_Ir ( SEQ ID NO : 18 ) = left homology arm ( 983 bps )$$

$$10 DargR\_rf ( SEQ ID NO : 19 ) , \text{} + DargR\_rr ( SEQ ID NO : 20 ) = left homology arm ( 984 bps )$$

The PCR products were purified using the “QIAquick PCR Purification Kit” (Qiagen GmbH, Hilden, Germany).

The linearized plasmid and the PCR products were then assembled using the “NEBuilder HiFi DNA Assembly Cloning Kit” (New England BioLabs Inc., Ipswich, USA, Cat. No. E5520). The resulting deletion vector was named pK18mobsacB_DargR. It was verified by restriction enzyme digestion and DNA sequencing.

For deleting the argR gene, pK18mobsacB_DargR was transformed into Corynebacterium glutamicum ATCC13032 (Kinoshita et al., J. Gen. Appl. Microbiol. 1957; 3(3): 193-205) by electroporation. Chromosomal integration (resulting from a first recombination event) was selected by plating on BHI agar supplemented with 134 g/I sorbitol, 2.5 g/I yeast extract and 25 mg/l kanamycin. The agar plates were incubated for 48 h at 33° C.

Individual colonies were transferred onto fresh agar plates (with 25 mg/l kanamycin) and incubated for 24 h at 33° C. Liquid cultures of these clones were cultivated for 24 h at 33° C. in 10 ml BHI medium contained in 100 ml Erlenmeyer flasks with 3 baffles. To isolate clones that have encountered a second recombination event, an aliquot was taken from each liquid culture, suitably diluted and plated (typically 100 to 200 μl) on BHI agar supplemented with 10% saccharose. These agar plates were incubated for 48 h at 33° C. Colonies growing on the saccharose containing agar plates were then examined for kanamycin sensitivity. To do so a toothpick was used to remove cell material from the colony and to transfer it onto BHI agar containing 25 mg/l kanamycin and onto BHI agar containing 10% saccharose. The agar plates were incubated for 60 h at 33° C. Clones that proved to be sensitive to kanamycin and resistant to saccharose were examined by PCR and DNA sequencing. The resulting strain was named ATCC13032_DargR.

### Example 6: Transformation of C. glutamicum ATCC13032_DargR with the Expression Plasmids

Strain ATCC13032_DargR was transformed by electroporation with the expression plasmids pLIB_P_AGAT-Mp, pLIB_serE_AGAT-Mp, pLIB_thrE_AGAT-Mp and pLIB_NCgl2566_AGAT-Mp. Plasmid containing cells were selected with 25 mg/l kanamycin. The resulting plasmid containing strains are shown in Table 6.

TABLE 6
Plasmid containing derivatives of C. glutamicum ATCC13032

| Recipient strain | Plasmid | Resulting strain |
|---|---|---|
| ATCC13032_DargR | pLIB_P_AGAT-Mp | ATCC13032_DargR/pLIB_P_AGAT-Mp |
| ATCC13032_DargR | pLIB_serE_AGAT-Mp | ATCC13032_DargR/pLIB_serE_AGAT-Mp |
| ATCC13032_DargR | pLIB_thrE_AGAT-Mp | ATCC13032_DargR/pLIB_thrE_AGAT-Mp |
| ATCC13032_DargR | pLIB_NCgl2566_AGAT- | ATCC13032_DargR/pLIB_NCgl2566_AGAT-Mp |
| | Mp | |

### Example 7: Impact of Overexpression of Transporter Genes on GAA Production

To assess the impact of the overexpression of the transporter genes on GAA production, strains ATCC13032_DargR/pLIB_P_AGAT-Mp, ATCC13032_DargR/pLIB_serE_AGAT-Mp ATCC13032_DargR/pLIB_thrE_AGAT-Mp, and ATCC13032_DargR/pLIB_NCgl2566_AGAT-Mp were cultivated in the Wouter Duetz system in production medium and the resulting GAA titers were determined as described above.

TABLE 7
Impact of the overexpression of transporter genes on GAA production

| Strain | GAA |
|---|---|
| ATCC13032_DargR/pLIB_P_AGAT-Mp | 286 mg/l |
| ATCC13032_DargR/pLIB_serE_AGAT-Mp | 1009 mg/l |
| ATCC13032_DargR/pLIB_thrE_AGAT-Mp | 398 mg/l |
| ATCC13032_DargR/pLIB_NCgl2566_AGAT-Mp | 319 mg/l |

The cultivation of the strains ATCC13032_DargR/pLIB_serE_AGAT-MpATCC13032_DargR/pLIB_thrE_AGAT-Mp, and ATCC13032_DargR/pLIB_NCgl2566_AGAT-Mp resulted in higher GAA titers, compared to ATCC13032/pLIB_P_AGAT-Mp (see Table 7). We conclude that overexpression of the transporters improves the production of GAA.

1. A microorganism, comprising;
at least one heterologous or synthetic gene coding for a protein having a function of an L-arginine:glycine amidinotransferase and having an overexpressed gene coding for a transmembrane exporter protein, wherein the transmembrane exporter protein is selected from the group consisting of proteins of the Drug/Metabolite Transporter (DMT) superfamily (TC 2.A.7), from proteins of the 10 TMS Drug/Metabolite Exporter (DME) Family (TC 2.A.7.3), from proteins of the Threonine/Serine Exporter (ThrE) Family (TC 2.A.79), and from proteins of the Resistance to Homoserine/Threonine (RhtB) Family (TC 2.A.76).

2. The microorganism of claim 1, wherein the transmembrane exporter protein has an identity of at least 20% to a protein comprising an amino acid sequence according to SEQ ID NO:6 or to a protein comprising an amino acid sequence according to SEQ ID NO: 10 or to a protein comprising an amino acid sequence according to SEQ ID NO: 14.

3. The microorganism of claim 1, wherein the transmembrane exporter protein has an identity of at least 70% to a protein comprising an amino acid sequence according to SEQ ID NO:6 or to a protein comprising an amino acid sequence according to SEQ ID NO:10 or to a protein comprising an amino acid sequence according to SEQ ID NO: 14.

4. The microorganism of claim 2, wherein the transmembrane exporter protein is encoded by an open reading frame of gene NCgl0580 of Corynebacterium glutamicum according to Seq ID NO:5.

5. The microorganism of claim 2, wherein the transmembrane exporter protein is encoded by an open reading frame of gene NCgl2533 of Corynebacterium glutamicum according to Seq ID NO:9.

6. The microorganism of claim 2, wherein the transmembrane exporter protein is encoded by an open reading frame of gene NCgl2566 of Corynebacterium glutamicum according to SEQ ID NO:13.

7. The microorganism of claim 1, wherein an overexpression of agene coding for a transmembrane exporter protein is achieved by increasing a copy number of a gene and/or by functionally linking the gene with a strong promoter and/or by enhancing a ribosomal binding site and/or by codon usage optimization of a start codon or of a whole gene.

8. The microorganism of claim 1, wherein the microorganism has an increased ability to produce L-arginine from L-orithine compared with an ability of a wildtype microorganism.

9. The microorganism of claim 8, wherein an expression of an argR gene coding for an arginine responsive repressor protein ArgR is attenuated compared to an expression of an argR gene in the wildtype microorganism or wherein the argR gene is inactivated or deleted.

10. A method for a fermentative production of guanidino acetic acid (GAA), the method comprising:
cultivating the microorganism as defined in claim 1 in a medium, and
accumulating GAA in the medium to form a GAA containing fermentation broth.

11. The method of claim 10, further comprising:
isolating GAA from the GAA containing fermentation broth.

12. A The microorganism as claimed in claim 1, further comprising:
a gene coding for an enzyme having an activity of a guanidinoacetate N-methyltransferase.

13. The microorganism of claim 12, wherein the gene coding for an enzyme having the activity of a guanidinoacetate N-methyltransferase is overexpressed.

14. A method for a fermentative production of creatine, the method comprising:
cultivating the microorganism as defined in claim 12, and
accumulating creatine in the medium to form a creatine containing fermentation broth.

15. The method of claim 14, further comprising:
isolating creatine from the creatine containing fermentation broth.
